(19) United States
(12) Patent Application Publication
(10) Pub. No.: US 2022/0361297 A1
(43) Pub. Date: Nov. 10, 2022

(54) ADVANCED HEATING PAD

(71) Applicant: Sunbeam Products, Inc., Boca Raton, FL (US)

(72) Inventors: Amanda Barrett, Smyrna, TN (US); Marie Chenowith, Boynton Beach, FL (US); George Hardin, Hattiesburg, MS (US)

(21) Appl. No.: 17/662,779

(22) Filed: May 10, 2022

Related U.S. Application Data
(60) Provisional application No. 63/186,531, filed on May 10, 2021.

(51) Int. Cl. H05B 3/34; A61F 7/00; H05B 3/38; H05B 3/14

(52) U.S. Cl. CPC H05B 3/347; A61F 7/007; H05B 3/38; H05B 3/145; H05B 3/146; A61F 2007/0088

(57) ABSTRACT

A multi layer advanced heating pad comprising a reflective layer proximate to a heating layer and a black body a layer configured to concentrate and amplify heat emitted from the heating layer and reflected by the reflective layer.

### CROSS REFERENCE TO RELATED APPLICATIONS

This application claims priority to U.S. Application No. 63/186,531 filed May 10, 2021 and titled “ADVANCED HEATING PAD.” U.S. Application No. 63/186,531 is hereby fully incorporated by reference as if set forth fully herein.

### FIELD OF INVENTION

The invention refers to heating pads. More specifically, the invention includes heating pads that include a novel layer configuration to provide improved heating depth in users.

### BACKGROUND OF THE INVENTION

Clinical studies have shown that the application of heat can provide relief for muscle and joint pain. Chronic pain sufferers are open to using non-oral pain relief methods, such as heating pads. However, currently existing heating pads are constructed such that heat only penetrates a small depth below the user's skin. This lack of deep heating requires these heating pads to employ inefficient wasteful heating to treat certain types of ailments such as certain deep tissue ailments and/or limits the effectiveness of these heating pads to treat such ailments entirely. Some known heating pads have attempted to rectify these defects by using inserts that are mostly ineffective, expensive, and/or deform the heating pad so as to prevent the pad from easily conforming to a user's body.

In light of the foregoing, there is an ongoing need for an improved heating pad designed to provide reliable heating at deeper depths below a user's skin.

### SUMMARY

Embodiments described here relate to a heating pad comprising an anterior side and a posterior side. The heating pad also comprises a first layer on the posterior side and a second layer having a heating element embedded therein. The heating element is configured to selectively generate heat when active to increase the temperature of the heating pad. The heating pad further comprises a third layer positioned between the second layer and the anterior side. The third layer comprises a reflective material positioned to reflect at least a portion of the heat generated from the third layer towards the first layer and away from the anterior side. The heating pad also comprises a fourth layer on the anterior side. The first layer comprises a material configured to collect and concentrate the heat generated by the second layer including the portion reflected by the third layer.

In some embodiments of the heating pad, the material of the first layer includes graphene fabric. In some of these embodiments, the graphene fabric includes fabric impregnated with graphene powder. Additionally or alternatively, in some embodiments, the graphene fabric includes a fabric material interwoven with graphene.

In some embodiments of the heating pad, the material of the first layer includes polyester material having a plurality of cavities arranged in a grid pattern that contain at least one glass bead.

In some embodiments, the heating pad further comprises a fifth layer disposed between the third layer and the fourth layer. In some of these embodiments, the fifth layer is comprised of polyester batting fiber. In some of these embodiments, the heating pad further comprises a sixth layer disposed between the first layer and the second layer. In some of these embodiments, the fifth layer and the sixth layer are comprised of polyester batting fiber.

In some embodiments of the heating pad, the second layer is comprised of a scrim and the heating element includes resistive heating wire supported by the scrim. In some of these embodiments, the reflective material includes insulbrite directly in contact with the scrim.

In some embodiments of the heating pad, the fourth layer is comprised of microfleece material.

In some embodiments, the heating pad further comprises a power source and a temperature controller coupled to the heating element and the power source. In these embodiments, the temperature controller is configured to supply power to the heating element from the power source based on a selected operational setting.

In some embodiments of the heating pad, the material of the first layer has an emissivity value above 0.9.

In some embodiments of the heating pad, the heating element includes sensing wire.

Additionally or alternatively, in some embodiments, the heating element includes a positive temperature coefficient (PTC) heater.

In some embodiments of the heating pad, when the heating element is active and the posterior side is contacted with a skin layer of a user, the first layer, the second layer, and the third layer combine to generate far infrared heating that produces a temperature value greater than or equal to 104 degrees Fahrenheit in a first muscle layer of the user after a first time period in the range between approximately 37 minutes and approximately 47 minutes. In some of these embodiments, the far infrared heating produces a temperature value greater than or equal to 104 degrees Fahrenheit in a second muscle layer of the user after a second time period in the range between approximately 38 minutes and approximately 50 minutes. Furthermore, in some of these embodiments, the far infrared heating produces a temperature value greater than or equal to 104 degrees Fahrenheit in a third muscle layer of the user after a third time period in the range between approximately 60 minutes and approximately 70 minutes.

Embodiments described here also relate to a heating pad comprising an anterior side, a posterior side, an outer fabric layer on the anterior side, and a first batting layer below the outer fabric layer. The heating pad also comprises a heat element layer having a heating element embedded therein. The heating element is configured to be selectively generate heat to increase the temperature of the heating pad according to a selected operational setting when connected to a power source. The heating pad also comprises a reflective layer between the first batting layer and the heat element layer. The reflective layer comprises a reflective material positioned to reflect at least a portion of the heat generated from the heat element layer towards the posterior side and away from the anterior side. The heating pad also comprises a second batting layer below the heat element layer and a black body layer on the posterior side that comprises a material configured to collect and concentrate the heat generated by the heat element layer including the portion reflected by the reflective layer.

In some embodiments of the heating pad the material of the black body layer includes graphene fabric. Additionally or alternatively, in some embodiments the material of the blackbody layer includes polyester material having a plurality of cavities arranged in a grid pattern that contain at least one glass bead.

In some embodiments, the heating pad further comprises a temperature controller coupled to the heating element and the power source. The temperature controller is configured to supply power to the heating element from the power source based on the selected operational setting.

### BRIEF DESCRIPTION OF THE DRAWINGS

For a better understanding of the various embodiments of the present invention, reference may be made to the accompanying drawings in which:

FIG. 1 is a front perspective view of a heating pad;

FIG. 2 is a schematic diagram of the layers in a first embodiment of a heating pad;

FIG. 3 is a schematic diagram of layers in a second embodiment of the heating pad;

FIG. 4 is a schematic diagram of the layers in a third embodiment of the heating pad;

FIG. 5 is a graph showing the temperature change at different depths below a user's skin after 30 minutes for various embodiments of the heating pad described herein and conventional know heating pads.

While the disclosure is susceptible to various modifications and alternative forms, specific embodiments thereof are shown by way of example in the drawings and will herein be described in detail. It should be understood, however, that the drawings and detailed description presented herein are not intended to limit the disclosure to the particular embodiment disclosed, but on the contrary, the intention is to cover all modifications, equivalents, and alternatives falling within the spirit and scope of the present disclosure.

### DETAILED DESCRIPTION OF THE DRAWINGS

The invention will now be described with reference to the drawing figures, in which like reference numerals refer to like parts throughout. For purposes of clarity in illustrating the characteristics of the present invention, proportional relationships of the elements have not necessarily been maintained in the drawing figures.

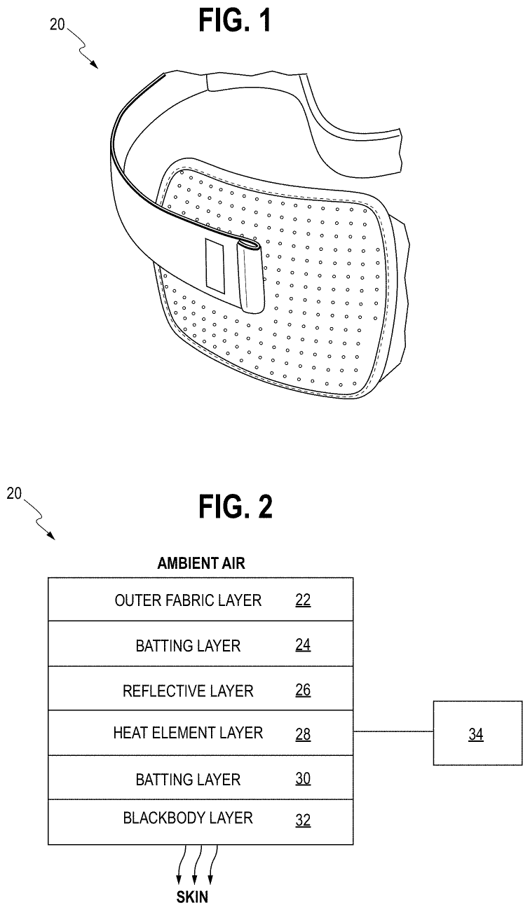

FIG. 1 shows a heating pad 20 according to disclosed embodiments. FIG. 2 shows a schematic diagram of the heating pad 20 according to disclosed embodiments. As seen in FIG. 2 the heating pad 20 can include a posterior side configured to contact a user's skin and an anterior side that can be exposed to ambient air during operation. Furthermore, the heating pad 20 can comprises an outer fabric layer 22 on the anterior side of the heating pad 20, a first batting layer 24 below the outer fabric layer 22, a reflective layer 26 below the first batting layer 24, a heat element layer 28 below the reflective layer 26 configured to heat the pad 20 to a selected temperature or according to a selected operational setting, a second batting layer 30, and a black body layer 32 below the second batting layer 30 on the posterior side of the heating pad 20. In some embodiments, the second batting layer 30 can be omitted.

In some embodiments, the reflective layer 26 can be configured to reflect heat radiated from the heating layer 28 back toward the black body layer 32 so as to decrease the amount of heat emitted on the anterior side of the heating pad 20. Furthermore, the black body layer 32 can be configured to have an emissivity value close to 1 such that the black body layer 32 concentrates the heat emitted from the heat element layer 28 and/or the heat reflected by the reflecting layer 28. In some embodiments, the emissivity can be configured to be approximately above 0.9. For example, the combination of layers described herein can generate an optimal far infrared heating system that penetrates heat deep into a user's tissue and in some particular embodiments enables heat to penetrate up to two times deeper than currently known heating pad systems. In certain embodiments, the system allows heat to penetrate tissue at a greater speed than existing systems that do not include the arrangement of reflective and black body materials as described herein. In certain embodiments, the heat penetrates at least 1.5 times or at least two times as fast as it does in known systems.

For example, in some embodiments, when the heating element of the heat element layer 28 is active and the posterior side is contacted with a skin layer of a user, the blackbody layer 32, the heat element layer 28, and the reflective layer 26 combine to generate the far infrared heating. In these embodiments, the far infrared heating can produce a temperature value greater than or equal to 104 degrees Fahrenheit in (1) a first muscle layer of the user after a first time period; (2) a second muscle layer of the user after a second time period; and (3) a third muscle layer after a third time period. The first muscle layer can include the muscle layer directly below a fat layer of the user, the second muscle layer can include the muscle layer below the first muscle layer, and the third muscle layer can include the muscle layer below second muscle layer. In some embodiments, the first time period can include a time range between approximately 37 minutes and approximately 47 minutes. In some embodiments, the second time period can include a time range between approximately 38 minutes and approximately 50 minutes. Furthermore, in some embodiments, the third time period can include a time range between approximately 60 minutes and approximately 70 minutes. Additional time ranges and specific values for achieving the temperature value greater than or equal to 104 degrees Fahrenheit at various muscle layers of the user are discussed below in connection with Table 1.

In some embodiments, the heating pad 20 can include a power source and temperature controller 34 coupled to the heat element layer 28. Various embodiments for the power source are contemplated including but not limited to a portable battery power source and a standard AC wall outlet. When operated, the temperature controller 34 is configured to supply power to the heating element from the power source based on a selected operational setting or temperature value. For example, in some embodiments, the selected operational setting can include a low setting, a medium setting, and a high setting. The medium setting can cause the temperature controller 34 to heat the heat element layer 28 to a temperature value or range of values that is higher than under the low setting but lower than under the high setting. In some embodiments, the high setting can be a maximum temperature setting for the heating pad 20.

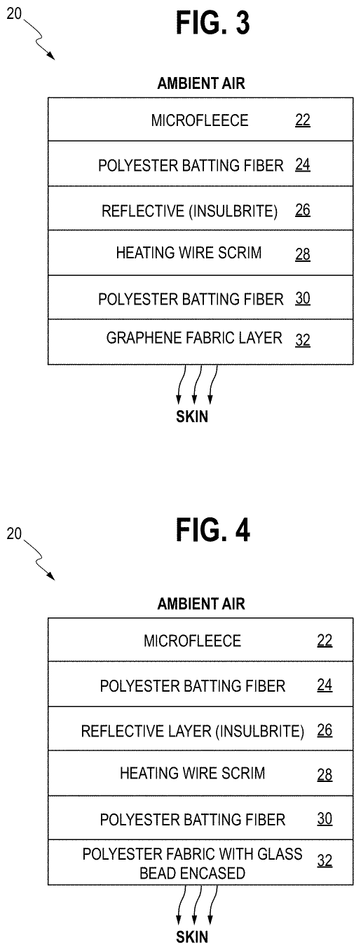

FIG. 3 depicts one example embodiment for the heating pad 20. As seen in FIG. 3, the outer fabric layer 22 can include a microfleece fabric and the first and second batting layers 24 and 30 can include polyester batting fiber. The heating element layer 28 can be comprised of a resistance based heating wire sewn to a scrim fabric and the reflective layer 26 can include insulbrite material directly in contact with the scrim fabric of the heating element layer 26. Some available alternatives to the resistance based heating wire can include sensing wire, positive temperature coefficient (PTC) heaters and other known structures. However, current testing data indicates that the resistance based heating wire performed best when combined with the other layers of the heating pad 20 described herein.

Furthermore, as seen in FIG. 3, in some embodiments, the black body layer 24 can be composed of a graphene material configured to collect and concentrate the heat emitted from the heat element layer 28 and/or the heat redefected by the reflecting layer 26 as discussed above. In some embodiments, the graphene material can include a fabric impregnated with graphene powder and/or a fabric material interwoven with graphene. Additionally or alternatively, in some embodiments such as shown in FIG. 4, the black body layer 32 can comprise a polyester fabric layer having a plurality of cavities that enclose at least one glass bead. Furthermore, in some embodiments, the black body layer 32 can include a combination of a PVC/vinyl material with a mesh covering.

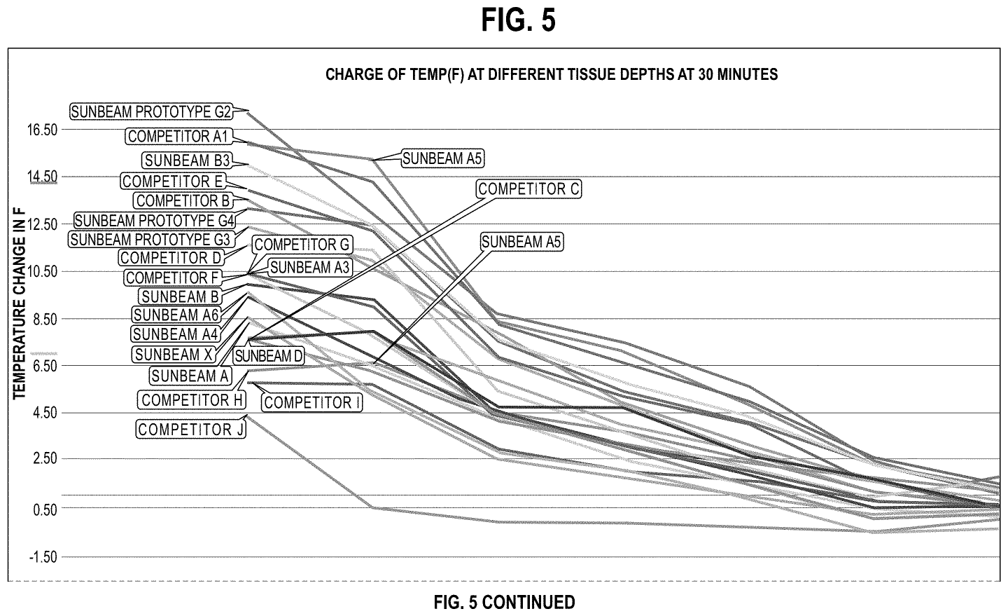

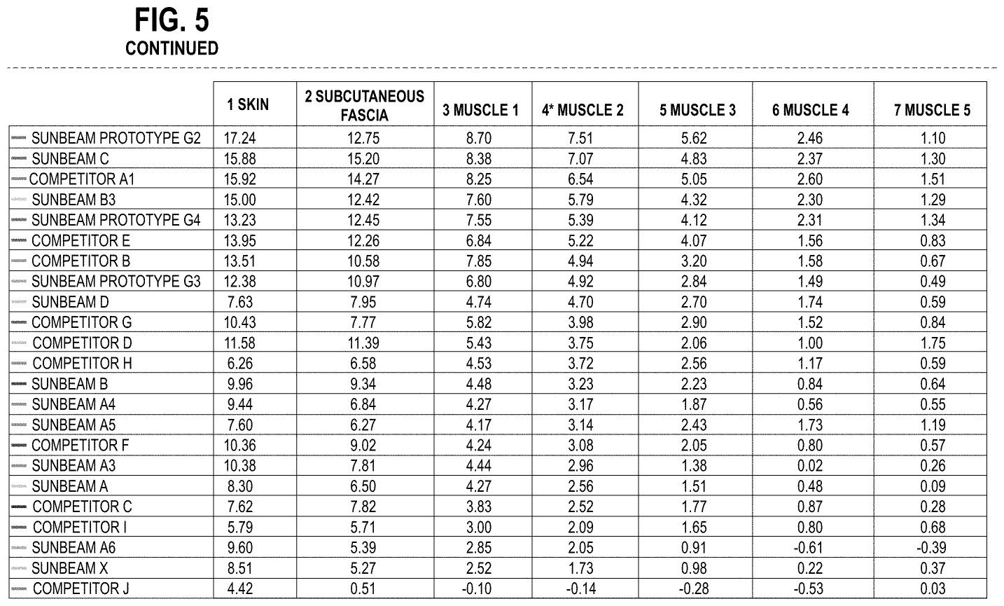

FIG. 5 shows a graph of temperature changes at different depths below a user's skin after 30 minutes for various embodiments of the heating pad 20 described herein compared with conventional know heating pads. As seen in FIG. 5, the embodiments of the heating pad 20 employing the graphene material in the black body layer 24 produce larger temperature changes than alternative conventional heating pads. Furthermore, the temperature depth performance advantage of the various embodiments of the heating pad 20 can be seen in table 1 below. Table 1 indicates the time each heating pad took to reach approximately 104 degrees F. at various depth levels below a user's skin. In instances where the temperature value at a specific depth did not reach 104 degrees F., the maximum measured temperature at that depth is recorded. Furthermore, as indicated by the text in the first column some of the maximum recorded temperature readings were taken after 30 minutes of testing. The remaining maximum temperature values were recorded after several hours of testing. As seen from table 1, the embodiments of the heating pad 20 employing the graphene material in the black body layer 32 (Sunbeam Prototypes G1, G2, G3, and G4) reliably reach 104 degrees F. at greater depths than alternative conventional heating pads.

TABLE 1

| | | avg | Time in min to reach above 104 F. *( ) indicates max temp reached | | | | | | |
|---|---|---|---|---|---|---|---|---|---|
| Pad | Setting | temp | Skin | fat | m1 | m2 | m3 | m5 | m7 |
| Sunbeam | high | 112.79 | 15.49 | 27.00 | 44.40 | 49.53 | 65.94 | *(103.14) | *(100.74) |
| Prototype G2 | | | | | | | | | |
| Sunbeam | high | 111.10 | 11.87 | 22.74 | 41.50 | 43.56 | 69.76 | *(102.58) | *(100.57) |
| Prototype G1 | | | | | | | | | |
| Sunbeam | high | 131.03 | 27.60 | 29.11 | 46.19 | 67.17 | *(103.05) | *(101.10) | *(100.42) |
| Prototype G3 | | | | | | | | | |
| Sunbeam | high | 102.87 | 26.71 | 31.84 | *(101.11) | *(99.62) | *(98.87) | *(98.33) | *(98.31) |
| Prototype G4 | | | | | | | | | |
| [30 min] | | | | | | | | | |
| Sunbeam | low | 109.69 | 23.21 | 54.84 | *(103.69) | *(103.23) | *(101.56) | *(100.53) | *(99.62) |
| Prototype G2 | | | | | | | | | |
| Sunbeam | med. | 120.76 | 5.57 | 16.03 | 37.95 | 38.77 | 61.55 | *(103.29) | *(101.07) |
| Prototype G1 | | | | | | | | | |
| Sunbeam A5 | high | 105.3 | 60.35 | 57.40 | *(103.85) | *(102.79) | *(101.49) | *(100.33) | *(99.77) |
| Sunbeam A4 | high | 107.5 | 46.67 | 65.28 | *(103.53) | *(102.50) | *(100.92) | *(99.71) | *(99.38) |
| Sunbeam A3 | high | 105.5 | 64.04 | 72.32 | *(103.44) | *(102.41) | *(101.15) | *(99.88) | *(99.55) |
| Competitor pad A1 | temp | 120.3 | 12.89 | 11.95 | *(103.49) | *(102.17) | *(100.95) | *(99.28) | *(98.73) |
| [30 min] | | | | | | | | | |
| Competitor pad E | high | 119 | 21.47 | 23.90 | *(102.34) | *(101.16) | *(100.35) | *(99.10) | *(98.78) |
| [30 min] | | | | | | | | | |
| Competitor pad B | high | 128.7 | 26.32 | *(102.73) | *(101.76) | *(100.42) | *(99.67) | *(99.06) | *(99.02) |
| [30 min] | | | | | | | | | |
| Competitor pad J | high | 90.50 | *(96.59) | *(95.18) | *(96.04) | *(96.40) | *(96.84) | *(97.54) | *(98.10) |
| [30 min] | | | | | | | | | |
| Competitor pad G | high | 100.6 | *(102.07) | *(101.21) | *(100.33) | *(99.620) | *(99.24) | *(98.82) | *(98.99) |
| [30 min] | | | | | | | | | |
| Competitor pad D | high | 104 | 31.92 | 31.67 | *(100.96) | *(100.09) | *(98.90) | *(98.07) | *(97.81) |
| [30 min] | | | | | | | | | |
| Competitor pad F | high | 119.7 | *(102.78) | *(102.30) | *(100.25) | *(99.60) | *(99.04) | *(98.63) | *(98.62) |
| [30 min] | | | | | | | | | |

From the foregoing, it will be seen that the various embodiments of the present invention are well adapted to attain all the objectives and advantages hereinabove set forth together with still other advantages which are obvious and which are inherent to the present structures. It will be understood that certain features and sub-combinations of the present embodiments are of utility and may be employed without reference to other features and sub-combinations. Since many possible embodiments of the present invention may be made without departing from the spirit and scope of the present invention, it is also to be understood that all disclosures herein set forth or illustrated in the accompanying drawings are to be interpreted as illustrative only and not limiting. The various constructions described above and illustrated in the drawings are presented by way of example only and are not intended to limit the concepts, principles and scope of the present invention.

Many changes, modifications, variations and other uses and applications of the present invention will, however, become apparent to those skilled in the art after considering the specification and the accompanying drawings. All such changes, modifications, variations and other uses and applications which do not depart from the spirit and scope of the invention are deemed to be covered by the invention which is limited only by the claims which follow.

What is claimed is:

1. A heating pad comprising:
an anterior side;
a posterior side;
a first layer on the posterior side;
a second layer having a heating element embedded therein, the heating element being configured to be selectively generate heat when active to increase the temperature of the heating pad;
a third layer positioned between the second layer and the anterior side, the third layer comprising a reflective material positioned to reflect at least a portion of the heat generated from the third layer towards the first layer and away from the anterior side; and
a fourth layer on the anterior side,
wherein the first layer comprises a material configured to collect and concentrate the heat generated by the second layer including the portion reflected by the third layer.

2. The heating pad of claim 1, wherein the material of the first layer includes graphene fabric.

3. The heating pad of claim 2, wherein the graphene fabric includes fabric impregnated with graphene powder.

4. The heating pad of claim 2, wherein the graphene fabric includes a fabric material interwoven with graphene.

5. The heating pad of claim 1, wherein the material of the first layer includes polyester material having a plurality of cavities arranged in a grid pattern that contain at least one glass bead.

6. The heating pad of claim 1 further comprising a fifth layer disposed between the third layer and the fourth layer and a sixth layer disposed between the first layer and the second layer.

7. The heating pad of claim 6, wherein the fifth layer and the sixth layer are comprised of polyester batting fiber.

8. The heating pad of claim 1 wherein when the heating element is active and the posterior side is contacted with a skin layer of a user, the first layer, the second layer, and the third layer combine to generate far infrared heating that produces a temperature value greater than or equal to 104 degrees Fahrenheit in a first muscle layer of the user after a first time period in the range between approximately 37 minutes and approximately 47 minutes.

9. The heating pad of claim 8 wherein the far infrared heating produces a temperature value greater than or equal to 104 degrees Fahrenheit in a second muscle layer of the user after a second time period in the range between approximately 38 minutes and approximately 50 minutes.

10. The heating pad of claim 8 wherein the far infrared heating produces a temperature value greater than or equal to 104 degrees Fahrenheit in a third muscle layer of the user after a third time period in the range between approximately 60 minutes and approximately 70 minutes.

11. The heating pad of claim 1, wherein the second layer is comprised of a scrim and the heating element includes resistive heating wire supported by the scrim, and wherein the reflective material includes insulbrite directly in contact with the scrim

12. The heating pad of claim 1, wherein the fourth layer is comprised of microfleece material.

13. The heating pad of claim 1 further comprising:
a power source; and
a temperature controller coupled to the heating element and the power source,
wherein the temperature controller is configured to supply power to the heating element from the power source based on a selected operational setting.

14. The heating pad of claim 1, wherein the material of the first layer has an emissivity value above 0.9.

15. The heating pad of claim 1, wherein the heating element includes sensing wire.

16. The heating pad of claim 1, wherein the heating element includes a positive temperature coefficient (PTC) heater.

17. A heating pad comprising:
an anterior side;
a posterior side;
an outer fabric layer on the anterior side;
a first batting layer below the outer fabric layer;
a heat element layer having a heating element embedded therein, the heating element being configured to be selectively generate heat to increase the temperature of the heating pad according to a selected operational setting when connected to a power source;
a reflective layer between the first batting layer and the heat element layer, the reflective layer comprising a reflective material positioned to reflect at least a portion of the heat generated from the heat element layer towards the posterior side and away from the anterior side;
a second batting layer below the heat element layer;
a black body layer on the posterior side that comprises a material configured to collect and concentrate the heat generated by the heat element layer including the portion reflected by the reflective layer.

18. The heating pad of claim 17, wherein the material of the black body layer includes graphene fabric.

19. The heating pad of claim 17, wherein the material of the blackbody layer includes polyester material having a plurality of cavities arranged in a grid pattern that contain at least one glass bead.

20. The heating pad of claim 17 further comprising a temperature controller coupled to the heating element and the power source, wherein the temperature controller is configured to supply power to the heating element from the power source based on the selected operational setting.
